(19) United States
(12) Patent Application Publication
(10) Pub. No.: US 2019/0216347 A1
(43) Pub. Date: Jul. 18, 2019

(54) SYSTEMS AND METHODS FOR ACTIVATION MAPPING OF THE HEART WITHOUT THE USE OF A REFERENCE CATHETER

(71) Applicant: Boston Scientific Scimed Inc., Maple Grove, MN (US)

(72) Inventors: Alireza Ghodrati, Hopkinton, MA (US); Nathan H. Bennett, Cambridge, MA (US); Brian Stewart, North Reading, MA (US)

(21) Appl. No.: 16/249,165

(22) Filed: Jan. 16, 2019

Related U.S. Application Data
(60) Provisional application No. 62/617,912, filed on Jan. 16, 2018.

(51) Int. Cl. A61B 5/04; A61B 5/042; A61B 5/044; A61B 5/06; A61B 5/00

(52) U.S. Cl. CPC A61B 5/04011; A61B 5/04017; A61B 5/0422; A61B 2562/0209; A61B 5/068; A61B 5/6858; A61B 5/7203; A61B 5/044

(57) ABSTRACT

A mapping system including a catheter including multiple, spatially distributed electrodes to measure electrical signals of a heart, a system to determine a position of the electrodes in the heart at multiple, different catheter positions in the heart, and a processing unit. The processing unit to receive the measured electrical signals from each position of the multiple, different catheter positions, determine velocity vectors based on the measured electrical signals, and to calculate, from the velocity vectors, an activation time at vertices of a mesh provided by the processing unit as a geometry of the heart.

### CROSS-REFERENCE TO RELATED APPLICATION

This application claims priority to Provisional Application No. 62/617,912, filed Jan. 16, 2018, which is herein incorporated by reference in its entirety.

### TECHNICAL FIELD

The present disclosure relates to medical systems and methods for mapping electrical activity in the body. More specifically, the disclosure relates to systems and methods for mapping electrical activity in the heart.

### BACKGROUND

Use of minimally invasive procedures, such as catheter ablation, to treat a variety of heart conditions, such as supraventricular and ventricular arrhythmias, is becoming increasingly more prevalent. Such procedures involve the mapping of electrical activity in the heart based on cardiac signals, such as at various locations on the endocardium surface, referred to as cardiac mapping, to identify the site of origin of the arrhythmia followed by a targeted ablation of the site. To perform such cardiac mapping a catheter with one or more electrodes can be inserted into the patient's heart chamber.

Conventional three-dimensional (3D) mapping techniques include contact mapping and non-contact mapping, and may employ a combination of contact and non-contact mapping. In these techniques, one or more catheters are advanced into the heart. With some catheters, once in the chamber, the catheter may be deployed to assume a 3D shape. In contact mapping, physiological signals resulting from the electrical activity of the heart are acquired with one or more electrodes located at the catheter distal tip after determining that the tip is in stable and steady contact with the endocardium surface of a particular heart chamber. In non-contact-based mapping systems, using the signals detected by the non-contact electrodes and information on chamber anatomy and relative electrode location, the system provides physiological information regarding the endocardium of the heart chamber. Location and electrical activity is usually measured sequentially on a point-by-point basis at about 50 to 200 points on the internal surface of the heart to construct an electro-anatomical depiction of the heart. The generated map may then serve as the basis for deciding on a therapeutic course of action, for example, tissue ablation, to alter the propagation of the heart's electrical activity and to restore normal heart rhythm.

In cardiac mapping of organized arrhythmias, such as atrial tachycardia, the electrical activation pattern in the heart is repeatable over time, and electrical activity measured by a catheter can be associated with electrical activity measured by a reference catheter or electrode, such as a coronary sinus (CS) reference catheter or electrode. As a result, sequential activation mapping of organized arrhythmias in the heart is possible.

In conventional sequential activation mapping, a mapping catheter measures electrical activity at a number of different locations in the heart with respect to the electrical activity at a reference catheter, such as a CS catheter. Without data from a reference catheter, the temporal relationships between electrical activations at different locations are unknown. Thus, if the reference catheter moves during mapping, the mapping data measured before and after the reference catheter moves may not be able to be combined and most often the activation mapping will need to be repeated. Also, in some situations, the electrical activity at the reference catheter's location may be disorganized, fractionated, or dissociated, such that using data from the reference catheter may impose error into the calculations of the activation map.

### SUMMARY

In an Example 1, a mapping system including a catheter including multiple, spatially distributed electrodes configured to measure electrical signals of a heart, a system configured to determine a position of the electrodes in the heart at multiple, different catheter positions in the heart, and a processing unit, The processing unit to receive the measured electrical signals from each position of the multiple, different catheter positions, determine velocity vectors based on the measured electrical signals, and calculate, from the velocity vectors, an activation time at vertices of a mesh provided by the processing unit as a geometry of the heart.

In an Example 2, the mapping system of Example 1, wherein to calculate, from the velocity vectors, an activation time at vertices of a mesh, the processing unit interpolates the velocity vectors of each position of the multiple, different catheter positions to the vertices of the mesh.

In an Example 3, the mapping system of any of Examples 1 and 2, wherein to calculate, from the velocity vectors, an activation time at vertices of a mesh, the processing unit creates a linear system of equations for the vertices of the mesh.

In an Example 4, the mapping system of Example 3, wherein to create a linear system of equations for the vertices of the mesh, the processing unit determines equations for neighboring vertices of the mesh, which relate activation times of neighboring vertices to each other.

In an Example 5, the mapping system of Example 4, wherein to create a linear system of equations for the vertices of the mesh, the processing unit combines the equations for neighboring vertices of the mesh to create the linear system of equations.

In an Example 6, the mapping system of any of Examples 3-5, wherein to calculate, from the velocity vectors, an activation time at vertices of a mesh, the processing unit solves the linear system of equations using an overdetermined matrix of the linear system of equations.

In an Example 7, the mapping system of any of Examples 3-5, wherein to calculate, from the velocity vectors, an activation time at vertices of a mesh, the processing unit solves the linear system of equations iteratively by finding activation times of each of the neighboring vertices of a vertex using corresponding equations for the vertices from the linear system of equations, wherein at each of the iterations, the processing unit calculates the activation times of one neighboring layer of vertices of the vertex.

In an Example 8, the mapping system of any of Examples 1-7, wherein to determine velocity vectors based on the measured electrical signals, the processing unit is configured to determine relative activation times of the measured electrical signals at the position, fit a polynomial to the relative activation times, and calculate the velocity vectors based on the polynomial.

In an Example 9, the mapping system of any of Examples 1-8, comprising displaying at least some of the activation times on a map of an endocardial surface of the heart.

In an Example 10, a method of mapping electrical activity in a heart, including: measuring electrical signals of the heart via a catheter including multiple, spatially distributed electrodes; determining a position of the electrodes at multiple, different catheter positions in the heart; measuring the electrical signals at each position of the multiple, different catheter positions; determining, via a processing unit, velocity vectors based on the measured electrical signals; and calculating, via a processing unit and from the velocity vectors, an activation time at vertices of a mesh provided by the processing unit as a geometry of the heart.

In an Example 11, the method of Example 10, including interpolating the velocity vectors of each position of the multiple, different catheter positions to the vertices of the mesh and creating a linear system of equations for the vertices of the mesh.

In an Example 12, the method of Example 11, wherein creating a linear system of equations for the vertices of the mesh includes determining equations for neighboring vertices of the mesh, which relate activation times of neighboring vertices to each other and combining the equations for neighboring vertices of the mesh to create the linear system of equations.

In an Example 13, the method of any of Examples 11 and 12, wherein calculating, via a processing unit and from the velocity vectors, an activation time at vertices of a mesh includes solving the linear system of equations using an overdetermined matrix of the linear system of equations.

In an Example 14, a method of mapping electrical activity in a heart, comprising: measuring electrical signals of the heart via a catheter including multiple, spatially distributed electrodes; determining a position of the electrodes at multiple, different catheter positions in the heart; measuring the electrical signals at each position of the multiple, different catheter positions; determining, via a processing unit, velocity vectors based on the measured electrical signals; interpolating the velocity vectors to vertices of a mesh provided by the processing unit as a geometry of the heart; creating, via the processing unit, a linear system of equations for the vertices of the mesh; and calculating an activation time at each of the vertices of the mesh by solving the linear system of equations. The linear systems of equations created by: determining equations that relate activation times of neighboring vertices of the mesh to each other; and combining the equations for neighboring vertices of the mesh to create the linear system of equations.

In an Example 15, the method of Example 14, comprising displaying at least some of the activation times on a map of the heart to visualize the at least some of the activation times on an endocardial surface of the heart.

In an Example 16, a mapping system, including a catheter including multiple, spatially distributed electrodes configured to measure electrical signals of a heart, a system configured to determine a position of the electrodes in the heart at multiple, different catheter positions in the heart; and a processing unit. The processing unit to receive the measured electrical signals from each position of the multiple, different catheter positions, determine velocity vectors based on the measured electrical signals, and calculate, from the velocity vectors, an activation time at vertices of a mesh provided by the processing unit as a geometry of the heart.

In an Example 17, the mapping system of Example 16, wherein to calculate, from the velocity vectors, an activation time at vertices of a mesh, the processing unit interpolates the velocity vectors of each position of the multiple, different catheter positions to the vertices of the mesh.

In an Example 18, the mapping system of Example 16, wherein to calculate, from the velocity vectors, an activation time at vertices of a mesh, the processing unit creates a linear system of equations for the vertices of the mesh.

In an Example 19, the mapping system of Example 18, wherein to create a linear system of equations for the vertices of the mesh, the processing unit determines equations for neighboring vertices of the mesh, which relate activation times of neighboring vertices to each other.

In an Example 20, the mapping system of Example 19, wherein to create the linear system of equations for the vertices of the mesh, the processing unit combines the equations for neighboring vertices of the mesh to create the linear system of equations.

In an Example 21, the mapping system of Example 18, wherein to calculate, from the velocity vectors, an activation time at vertices of a mesh, the processing unit solves the linear system of equations using an overdetermined matrix of the linear system of equations.

In an Example 22, the mapping system of Example 18, wherein to calculate, from the velocity vectors, an activation time at vertices of a mesh, the processing unit solves the linear system of equations iteratively by finding activation times of each of the neighboring vertices of a vertex using corresponding equations for the vertices from the linear system of equations, wherein at each of the iterations, the processing unit calculates the activation times of one neighboring layer of vertices of the vertex.

In an Example 23, the mapping system of Example 16, wherein to determine velocity vectors based on the measured electrical signals, the processing unit is configured to determine relative activation times of the measured electrical signals at the position, fit a polynomial to the relative activation times, and calculate the velocity vectors based on the polynomial.

In an Example 24, the mapping system of Example 23, wherein to determine relative activation times of the measured electrical signals at the position, the processing unit is configured to select beats based on the position of the electrodes in the heart, pre-process the beats selected via low pass filtering and determining absolute value, cross-correlate pre-processed beats to neighboring pre-processed beats, and determine the relative activation times based on locations of maximum correlation. Also, wherein to fit a polynomial to the relative activation times, the processor is configured to project neighboring electrodes to a plane and fit a first order polynomial to the relative activation times.

In an Example 25, the mapping system of Example 16, wherein to determine velocity vectors based on the measured electrical signals, the processing unit is configured to determine whether the measured electrical signals at a position of the catheter are organized, and if the measured electrical signals at the position are organized the processing unit is configured to determine relative activation times of the measured electrical signals at the position, fit a polynomial to the relative activation times, and calculate velocity vectors based on the polynomial.

In an Example 26, the mapping system of Example 16, comprising displaying at least one of the velocity vectors and at least some of the activation times on a map of an endocardial surface of the heart.

In an Example 27, a method of mapping electrical activity in a heart, including measuring electrical signals of the heart via a catheter including multiple, spatially distributed electrodes, determining a position of the electrodes at multiple, different catheter positions in the heart, measuring the electrical signals at each position of the multiple, different catheter positions, determining, via a processing unit, velocity vectors based on the measured electrical signals, and calculating, via a processing unit and from the velocity vectors, an activation time at vertices of a mesh provided by the processing unit as a geometry of the heart.

In an Example 28, the method of Example 27, including interpolating the velocity vectors of each position of the multiple, different catheter positions to the vertices of the mesh and creating a linear system of equations for the vertices of the mesh.

In an Example 29, the method of Example 28, wherein creating a linear system of equations for the vertices of the mesh includes determining equations for neighboring vertices of the mesh, which relate activation times of neighboring vertices to each other and combining the equations for neighboring vertices of the mesh to create the linear system of equations.

In an Example 30, the method of Example 28, wherein calculating, via a processing unit and from the velocity vectors, an activation time at vertices of a mesh includes solving the linear system of equations using an overdetermined matrix of the linear system of equations.

In an Example 31, the method of Example 27, wherein determining velocity vectors based on the measured electrical signals includes determining relative activation times of the measured electrical signals at the position, fitting a polynomial to the relative activation times, and calculating the velocity vectors based on the polynomial.

In an Example 32, the method of Example 31, wherein determining relative activation times of the measured electrical signals at the position includes selecting beats based on the position of the electrodes in the heart, pre-processing the beats selected via low pass filtering and determining absolute value, cross-correlating pre-processed beats to neighboring pre-processed beats, and determining the relative activation times based on locations of maximum correlation. Also, wherein fitting a polynomial to the relative activation times includes projecting neighboring electrodes to a plane and fitting a first order polynomial to the relative activation times.

In an Example 33, a method of mapping electrical activity in a heart, comprising: measuring electrical signals of the heart via a catheter including multiple, spatially distributed electrodes; determining a position of the electrodes at multiple, different catheter positions in the heart; measuring the electrical signals at each position of the multiple, different catheter positions; determining, via a processing unit, velocity vectors based on the measured electrical signals; interpolating the velocity vectors to vertices of a mesh provided by the processing unit as a geometry of the heart; creating, via the processing unit, a linear system of equations for the vertices of the mesh, and calculating an activation time at each of the vertices of the mesh by solving the linear system of equations. The linear system of equations for the vertices of the mesh created by determining equations that relate activation times of neighboring vertices of the mesh to each other; and combining the equations for neighboring vertices of the mesh to create the linear system of equations.

In an Example 34, the method of Example 33, wherein determining velocity vectors based on the measured electrical signals includes: determining relative activation times of the measured electrical signals at the position; fitting a polynomial to the relative activation times; and calculating the velocity vectors based on the polynomial.

In an Example 35, the method of Example 33, comprising displaying the velocity vectors and at least some of the activation times on a map of the heart to visualize the velocity vectors and the at least some of the activation times on an endocardial surface of the heart.

While multiple embodiments are disclosed, still other embodiments of the disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating a cardiac mapping system, according to embodiments of the disclosure.

FIG. 2A is a diagram illustrating a perspective view of a catheter, according to embodiments of the disclosure.

FIG. 2B is a diagram illustrating an end view of the catheter, according to embodiments of the disclosure.

FIG. 2C is a diagram illustrating a side view of the catheter, according to embodiments of the disclosure.

FIG. 3 is a flow chart diagram illustrating a process for mapping and visualizing sensed electrical activity in the heart, according to embodiments of the disclosure.

FIG. 4 is a flow chart diagram illustrating a process to determine whether the measured electrical signals at one of the positions of the catheter are organized, according to embodiments of the disclosure.

FIG. 5A is a diagram illustrating an original electrogram of one of the measured electrical signals prior to pre-processing the measured electrical signal, according to embodiments of the disclosure.

FIG. 5B is a diagram illustrating a pre-processed electrogram, according to embodiments of the disclosure.

FIG. 6 is a flow chart diagram illustrating a process for determining relative activation times of the measured electrical signals and for determining vectors, such as propagation vectors and velocity vectors, at locations of the multiple, different catheter locations, according to embodiments of the disclosure.

FIG. 7 is a diagram illustrating a catheter having five electrodes on three different splines, which measure the electrical signals of a beat or beats of interest, according to embodiments of the disclosure.

FIG. 8 is a flow chart diagram illustrating a process for determining activation times from the velocity vectors, according to embodiments of the disclosure.

FIG. 9 is a diagram illustrating two neighboring points or vertices xi and xj on a mesh and a velocity vector vi at the point xi, according to embodiments of the disclosure.

FIG. 10 is a diagram illustrating neighboring points or vertices xi-xn and xp on a mesh and a velocity vector vi at the point xi, according to embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of systems and methods described herein facilitate processing sensed cardiac electrical signals to map and visualize organized electrical activity, such as atrial tachycardia, of a heart. The systems and methods provide mapping of the heart without using information gathered from a reference, such as a CS catheter. The systems and methods can use multi-electrode catheters to acquire multiple electrical signals at each position or location of the catheter in the heart. In embodiments, maps of the heart include improved visualization of organized electrical activity, including improved visualization of atrial tachycardia. The maps can include organizational aspects of the electrical activity in the heart, cycle lengths, velocity vectors, and activation times of the electrical activity on the endocardial surface of the heart. In embodiments, the systems and methods can also be used for mapping paced rhythms. In some embodiments, the systems and methods can be adapted for use in mapping reentrant rhythms.

According to embodiments, to perform aspects of embodiments of the methods described herein, cardiac electrical signals may be obtained from a mapping catheter that may be associated with a mapping system, a recording system, an ablation catheter, a memory device, such as a local memory and/or a cloud server, a communication component, a medical device, such as an implantable medical device, an external medical device, and/or a telemetry device, and/or the like.

As the term is used herein, a sensed cardiac electrical signal may refer to one or more sensed signals. Each cardiac electrical signal may include a number of intracardiac electrograms (EGMs) sensed within a patient's heart, and may include any number of features that may be ascertained by aspects of the system 100. Examples of cardiac electrical signal features include, but are not limited to, activation times, activations, activation waveforms, filtered activation waveforms, minimum voltage values, maximum voltages values, maximum negative time-derivatives of voltages, instantaneous potentials, voltage amplitudes, dominant frequencies, peak-to-peak voltages, and/or the like. A cardiac electrical signal feature may refer to one or more features extracted from one or more cardiac electrical signals, derived from one or more features that are extracted from one or more cardiac electrical signals, and/or the like. Additionally, a representation, on a cardiac and/or a surface map, of a cardiac electrical signal feature may represent one or more cardiac electrical signal features, an interpolation of a number of cardiac electrical signal features, and/or the like.

Each cardiac signal may be associated with a set of respective position coordinates that corresponds to the location at which the cardiac electrical signal was sensed. Each of the respective position coordinates for the sensed cardiac signals may include three-dimensional Cartesian coordinates, polar coordinates, and/or the like. In embodiments, other coordinate systems can be used. In embodiments, an arbitrary origin is used and the respective position coordinates refer to positions in space relative to the arbitrary origin. Since, in embodiments, the cardiac signals may be sensed on the cardiac surfaces, the respective position coordinates may be on the endocardial surface, epicardial surface, in the mid-myocardium of the patient's heart, and/or in the vicinity of one of these.

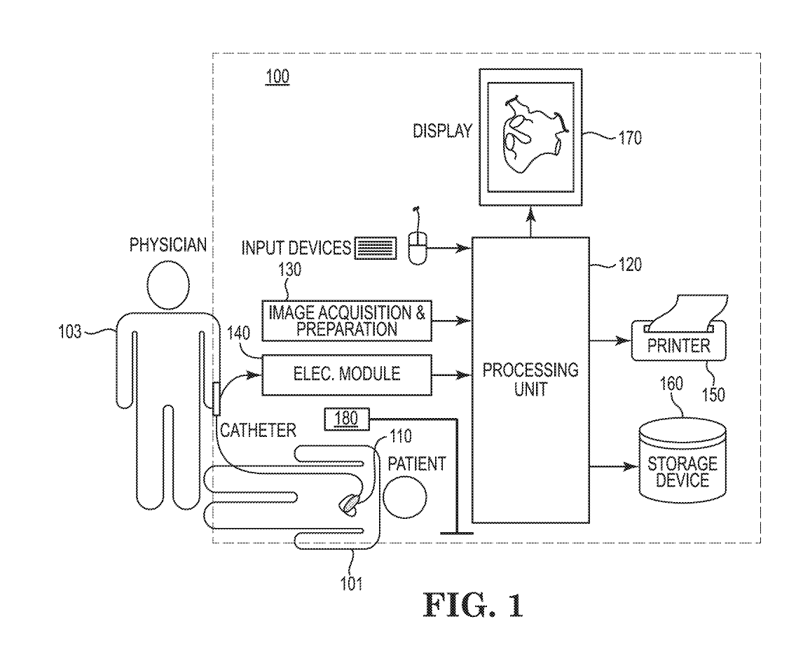

FIG. 1 is a diagram illustrating a cardiac mapping system 100, according to embodiments of the disclosure. As indicated above, embodiments of the subject matter disclosed herein may be implemented in a mapping system, such as mapping system 100, while other embodiments may be implemented in an ablation system, a recording system, a computer analysis system, and/or the like. The mapping system 100 includes a moveable catheter 110 having multiple, spatially distributed electrodes. During a signal-acquisition stage of a cardiac mapping procedure, the catheter 110 is displaced to multiple positions within the heart chamber into which the catheter 110 is inserted. In some embodiments, the distal end of the catheter 110 is fitted with multiple electrodes spread somewhat uniformly over the catheter. For example, the electrodes may be mounted on the catheter 110 following a 3D olive shape, a basket shape, and/or the like. The electrodes are mounted on a device capable of deploying the electrodes into the desired shape while inside the heart, and retracting the electrodes when the catheter is removed from the heart. To allow deployment into a 3D shape in the heart, electrodes may be mounted on a balloon, shape memory material such as Nitinol, actuable hinged structure, and/or the like. According to embodiments, the catheter 110 may be a mapping catheter, an ablation catheter, a diagnostic catheter, a CS catheter, and/or the like. For example, aspects of embodiments of the catheter 110, the electrical signals obtained using the catheter 110, and subsequent processing of the electrical signals, as described herein, may also be applicable in implementations having a recording system, ablation system, and/or any other system having a catheter with electrodes that may be configured to obtain cardiac electrical signals.

At each of the positions to which the catheter 110 is moved, the catheter's multiple electrodes acquire signals resulting from the electrical activity in the heart. Consequently, reconstructing and presenting to a user, such as a doctor and/or a technician, physiological data pertaining to the heart's electrical activity may be based on information acquired at multiple locations, thereby providing a more accurate and faithful reconstruction of physiological behavior of the endocardium surface. The acquisition of signals at multiple catheter positions in the heart chamber enables the catheter to effectively act as a “mega-catheter” whose effective number of electrodes and electrode span is proportional to the product of the number of locations in which signal acquisition is performed and the number of electrodes the catheter has.

To enhance the quality of the reconstructed physiological information at the endocardium surface, in some embodiments, the catheter 110 is moved to more than three locations, for example, more than 5, 10, or even 50 locations, within the heart chamber. Further, the spatial range over which the catheter is moved may be larger than one third (⅓) of the diameter of the heart cavity, for example, larger than 35%, 40%, 50% or even 60% of the diameter of the heart cavity. Additionally, in some embodiments, the reconstructed physiological information is computed based on signals measured over several heart beats, either at a single catheter location within the heart chamber or over several locations.

In some embodiments, where reconstructed physiological information is based on multiple measurements over several heart beats, the measurements may be synchronized with one another so that the measurements are performed at approximately the same phase of the heart cycle. The signal measurements over multiple beats may be synchronized based on features detected from physiological data such as surface electrocardiograms (ECGs) and/or intracardiac electrograms (EGMs).

The cardiac mapping system 100 further includes a processing unit 120 which performs several of the operations pertaining to the mapping procedure, including the reconstruction procedure to determine the physiological information at the endocardium surface and/or within a heart chamber. The processing unit 120 also may perform a catheter registration procedure. The processing unit 120 also may generate a 3D grid used to aggregate the information captured by the catheter 110 and to facilitate display of portions of that information.

The position of the catheter 110 inserted into the heart chamber can be determined using a conventional sensing and tracking system 180 that provides the 3D spatial coordinates of the catheter and/or its multiple electrodes with respect to the catheter's coordinate system as established by the sensing and tracking system. These 3D spatial locations may be used in building the 3D grid. Embodiments of the system 100 may use a hybrid location technology that combines impedance location with magnetic location technology. This combination may enable the system 100 to accurately track catheters that are connected to the system 100. Magnetic location technology uses magnetic fields generated by a localization generator positioned under the patient table to track catheters with magnetic sensors. Impedance location technology may be used to track catheters that may not be equipped with a magnetic location sensor, and may utilize surface ECG patches.

In embodiments, to perform a mapping procedure and reconstruct physiological information on the endocardium surface, the processing unit 120 may align the coordinate system of the catheter 110 with the endocardium surface's coordinate system. The processing unit 110 (or some other processing component of the system 100) may determine a coordinate system transformation function that transforms the 3D spatial coordinates of the catheter's locations into coordinates expressed in terms of the endocardium surface's coordinate system, and/or vice-versa. In embodiments, such a transformation may not be necessary, as embodiments of the 3D grid described herein may be used to capture contact and non-contact EGMs, and select mapping values based on statistical distributions associated with nodes of the 3D grid. The processing unit 120 also may perform post-processing operations on the physiological information to extract and display useful features of the information to the operator of the system 100 and/or other persons (e.g., a physician).

According to embodiments, the signals acquired by the multiple electrodes of catheter 110 are passed to the processing unit 120 via an electrical module 140, which may include, for example, a signal conditioning component. The electrical module 140 may be configured to receive the signals communicated from the catheter 110 and perform signal enhancement operations on the signals before they are forwarded to the processing unit 120. The electrical module 140 may include signal conditioning hardware, software, and/or firmware that may be used to amplify, filter and/or sample intracardiac potential measured by one or more electrodes. In some embodiments, the intracardiac signals have a maximum amplitude of 60 mV, with a mean of a few millivolts. In some embodiments, the signals are pre-processed in one or more of the electrical module 140 and the processing unit 120.

In some embodiments, the signals are bandpass filtered in a frequency range, e.g., 0.5-500 Hz, and sampled with analog to digital converters, e.g., with 15-bit resolution at 1 kHz. In some embodiments the signals are one or more of bandpass filtered in a frequency range, e.g., 0.5-500 Hz, derivatives of the filtered signals are obtained, absolute values of the derivatives are obtained, and the resulting waveforms are further low pass filtered, e.g., 20 Hz.

Other types of signal processing operations, such as spectral equalization, automatic gain control, etc. may also take place. For example, in embodiments, the intracardiac signals may be unipolar signals, measured relative to a reference, which may be a virtual reference, such as, for example, a Wilson's Central Terminal (WCT), from which the signal processing operations may compute differences to generate multipolar signals, e.g., bipolar signals, tripolar signals, etc. The signals may be otherwise processed, e.g., filtered, sampled, etc., before and/or after generating the multipolar signals. Also, to avoid interference with electrical equipment in the room, the signal may be filtered to remove the frequency corresponding to the power supply, e.g., 60 Hz. The resultant processed signals are forwarded by the module 140 to the processing unit 120 for further processing.

In embodiments, the processing unit 120 may be configured to process the resultant processed signals. In embodiments, because the processing unit 120 may be configured to process any number of different types of electrical signals, whether they have been preprocessed or not, the terms “electrical signal(s),” “cardiac electrical signal(s)” and terms including one or more of the aforementioned, shall be understood to refer to electrical signals, processed, e.g., pre-processed, electrical signals, raw signal data, interpolated electrical signals, estimated electrical signals, and/or any other type of information representing an electrical signal, as described herein.

Embodiments of the processing unit 120 may be configured to receive a number of electrical signals such as, for example, cardiac electrical signals. The processing unit 120 may receive the electrical signals from the electrical module 140, from a memory device, from a catheter, e.g., the catheter 110, from another computing device, from a user via a user input device, and/or the like. In embodiments, the processing unit 120 may receive an indication of a measurement location corresponding to each electrical signal. The processing unit 120 may be configured to generate, based on the electrical signals, a cardiac map, which may be presented via a display device 170. In embodiments, the cardiac map includes a number of annotations representing a number of cardiac signal features, which may include, for example, one or more activation times, minimum voltage values, maximum voltage values, maximum negative time-derivatives of voltage, instantaneous potentials, voltage amplitudes, dominant frequencies, and/or peak-to-peak voltages. In embodiments, the cardiac map may include one or more of organization information related to the organization of the electrical activity in the heart, cycle length information, and velocity vector information.

In some embodiments, the mapping system 100 includes a catheter 110 that includes multiple, spatially distributed electrodes configured to measure electrical signals of the heart, and a system, such as sensing and tracking system 180, configured to determine the positions of the electrodes of the catheter 110 in the heart at multiple, different catheter positions in the heart. The processing unit 120, and/or the electrical module 140, is configured to receive the measured electrical signals from each position of the multiple, different catheter positions for further processing. For example, for each electrode at or near the endocardium surface of the heart, such as within 2 or 3 millimeters of the endocardium surface, an interval of the measured electrical signals, such as 2.5 seconds, is selected for further processing. In some embodiments, the processing unit 120 is configured to determine velocity vectors based on the measured electrical signals, and interpolate the velocity vectors to vertices of a mesh provided by the processing unit as the geometry of the heart. Also, the processing unit 120 is configured to create a linear system of equations for the vertices of the mesh and to calculate an activation time at each of the vertices of the mesh by solving the linear system of equations.

In some embodiments, the processing unit 120 is configured to determine whether the measured electrical signals received from a position are organized before further processing, such as determining the velocity vectors based on the measured electrical signals, and interpolating the velocity vectors to vertices of a mesh provided by the processing unit as the geometry of the heart. In some embodiments, if the electrical activity at the position is organized, the processing unit 120 is configured to determine velocity vectors by determining relative activation times of the measured electrical signals at the position from neighboring electrodes, fit a polynomial to the relative activation times, and calculate the velocity vectors based on the polynomial. In some embodiments, the processing unit also calculates cycle lengths of the measured signals.

In some embodiments, the processing unit 120 is configured to facilitate display, via display device 170, of the cardiac map. In embodiments, the display may include any number of different types of parameters, settings, and/or the like that may be configured to change one or more features of an appearance of a displayed representation. For example, in embodiments, display parameters may include brightness, contrast, color saturation, sharpness, and/or the like. Thus, in embodiments, the map includes multiple colors and/or colored regions, where color saturation values, relative color saturation values, and/or the like, may be adjustable via user input, an algorithm, and/or the like. In some embodiments, the resulting map visualized on display 170 includes organization information for different positions in the heart, cycle lengths, velocity vectors characterizing the electrical activity on the endocardial surface of the heart, and activation times on the endocardial surface of the heart.

As further shown in FIG. 1, the cardiac mapping system 100 may include peripheral devices such as a printer 150 and/or the display device 170, both of which may be interconnected to the processing unit 120. Additionally, the mapping system 100 includes storage device 160 that may be used to store data acquired by the various interconnected modules, including the volumetric images, raw data measured by electrodes and/or the resultant endocardium representation computed therefrom, the partially computed transformations used to expedite the mapping procedures, the reconstructed physiological information corresponding to the endocardium surface, and/or the like.

In embodiments, the processing unit 120 may be configured to automatically improve the accuracy of its algorithms by using one or more artificial intelligence, i.e., machine-learning, techniques, classifiers, and/or the like. In embodiments, for example, the processing unit 120 may use one or more supervised and/or unsupervised techniques such as, for example, support vector machines (SVMs), k-nearest neighbor techniques, artificial neural networks, and/or the like. In embodiments, classifiers may be trained and/or adapted using feedback information from a user, other metrics, and/or the like.

The illustrative cardiac mapping system 100 shown in FIG. 1 is not intended to suggest any limitation as to the scope of use or functionality of embodiments of the present disclosure. Neither should the illustrative cardiac mapping system 100 be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the subject matter disclosed herein. For example, the electrical module 140 may be integrated with the processing unit 120. Additionally, or alternatively, aspects of embodiments of the cardiac mapping system 100 may be implemented in a computer analysis system configured to receive cardiac electrical signals and/or other information from a memory device, e.g., a cloud server, a mapping system memory, etc., and perform aspects of embodiments of the methods described herein for processing cardiac information, e.g., determining annotation waveforms, etc. That is, for example, a computer analysis system may include a processing unit 120, but not a mapping catheter.

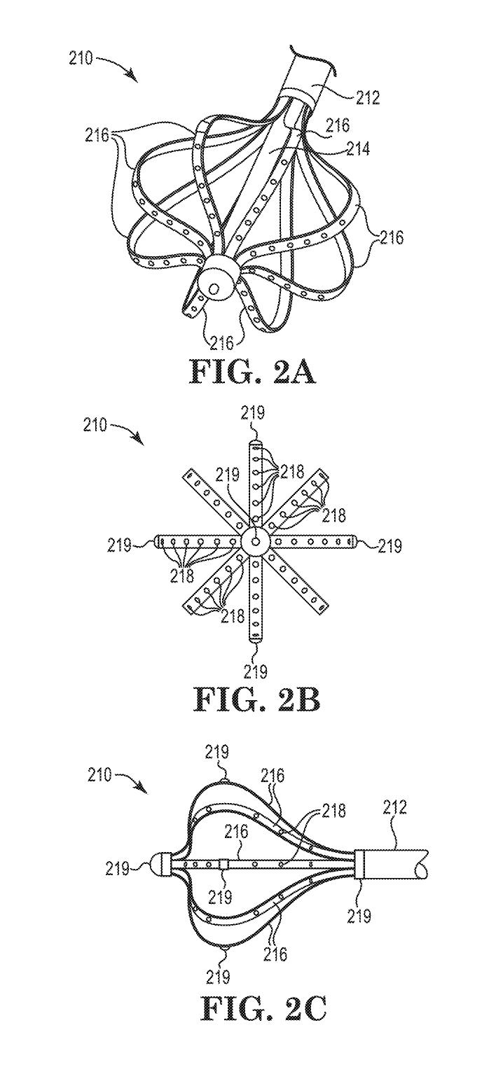

FIGS. 2A-2C are diagrams illustrating different views of a catheter 210, according to embodiments of the disclosure. FIG. 2A is a diagram illustrating a perspective view of the catheter 210, according to embodiments of the disclosure. FIG. 2B is a diagram illustrating an end view of the catheter 210, according to embodiments of the disclosure. FIG. 2C is a diagram illustrating a side view of the catheter 210, according to embodiments of the disclosure. In some embodiments, the catheter 210 is one example of catheter 110 (shown in FIG. 1).

Catheter 210 includes a base sleeve 212, a central retractable inner member 214, and multiple splines 216 connected to the base sleeve 212 at one end and the inner member 214 at the other end. When the inner member 214 is in an extended configuration or position (not shown), the splines 216 are pulled tight to the inner member 214 so that the catheter 210 has a narrow profile for guiding it through blood vessels. When the inner member 214 is retracted, as shown in FIGS. 2A-2C, the splines 216 are deployed and pushed into an outward “olive” shaped configuration for use in the heart cavity. The splines 216 each carry multiple electrodes, such that with the inner member 214 in the retracted configuration, the electrodes are deployed in the sense that they are distributed over a greater volume.

A large number of potential measuring electrodes (PME) 218 are mounted on the catheter 210 to measure electrical signals, such as cardiac electrical signals generated by the heart. Also, a number, such as greater than 6, current injecting electrodes (CIE) 219 are mounted on the catheter 210 to inject current into the heart. For example, 3 orthogonal CIE pairs may be mounted on the catheter 210, where each CIE pair defines a source and a sink electrode, respectively, for injecting current into the heart cavity.

It should be noted that a low impedance electrode can be used for current injection and in a case where many or all electrodes are capable of injecting current the designation of such electrodes as CIE on the catheter only indicates that these electrodes are actually being used for current injection. Also, it should be further appreciated that other configuration sets of CIE are possible as long as these configurations are known and can be accounted for. Examples of such configurations could be quadruples involving 4 CIE, or even a non-symmetrical configuration involving 3 CIE in known positions on the catheter. Also, configurations other than orthogonal pairs may be used for either method, and more than 2 CIE 219 may participate in current injection at a given time.

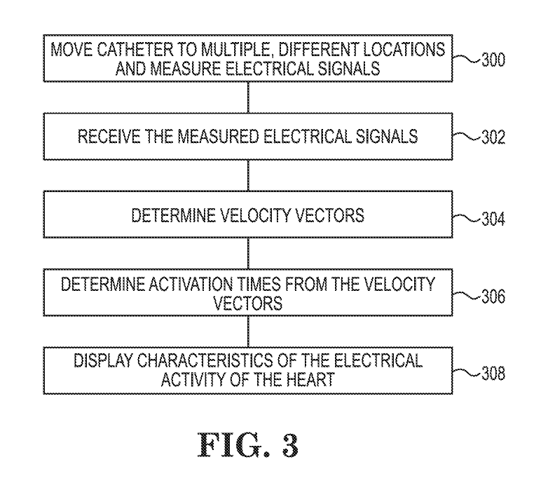

FIG. 3 is a flow chart diagram illustrating a process for mapping and visualizing sensed electrical activity in the heart, according to embodiments of the disclosure. The process can be performed by the cardiac mapping system 100 and used to map and visualize organized electrical activity, such as atrial tachycardia. The process is performed without using information gathered from a reference, such as a CS catheter.

The cardiac mapping system 100 includes the catheter 110, which has multiple, spatially distributed electrodes configured to measure electrical signals of the heart, and a sensing and tracking system, such as sensing and tracking system 180, that provides 3D spatial coordinates of the catheter 110 and its multiple electrodes with respect to the catheter's coordinate system. The sensing and tracking system is configured to determine the positions of the catheter electrodes in the heart at multiple, different catheter positions in the heart. In some embodiments, the cardiac mapping system 100 includes the catheter 210 of FIGS. 2A-2C.

The cardiac mapping system 100 also includes the processing unit 120 that provides processing power and electronic circuits for receiving and processing the measured electrical signals. In some embodiments, the processing unit 120 can determine a coordinate system transformation function that transforms the 3D spatial coordinates of the catheter and electrode locations in the catheter's coordinate system into coordinates expressed in terms of the endocardium surface's coordinate system. In some embodiments, the processing unit 120 includes the electrical module 140.

At 300, the catheter 110 is inserted into the heart of the patient and moved to multiple, different locations in the heart. The multiple, spatially distributed electrodes of the catheter 110, measure electrical signals generated by the heart at each of the multiple, different locations in the heart. The sensing and tracking system determines the locations of the electrodes in the heart at each of the multiple, different locations in the heart and the processing unit 120 determines the locations of the electrodes in the heart at each of the multiple, different locations in the heart in relation to the endocardium surface of the heart.

At 302, the processing unit 120, which may include the electrical module 140, receives the measured electrical signals acquired by the multiple electrodes of the catheter 110 at each location of the multiple, different catheter locations. In some embodiments, the measured electrical signals are assumed to be organized and the processing unit 120 further processes the measured electrical signals. In some embodiments, the processing unit 120 determines whether the measured electrical signals are organized at each of the multiple, different catheter locations, and proceeds with further processing of the measured electrical signals only if the measured electrical signals at the location of the multiple, different catheter locations are organized.

In some embodiments, the measured electrical signals are assumed to be organized and the processing unit 120 pre-processes the measured electrical signals prior to further processing of the signals. In some embodiments, in pre-processing, the processing unit 120 provides one or more of: the processing unit 120 low pass filters the measured electrical signals to filter out high frequency noise, the processing unit 120 determines the derivatives of the low pass filtered signal, the processing unit 120 determines an absolute value of the derivatives of the low pass filtered signal to make the processed signals positive, and then, optionally, the processing unit 120 low pass filters the derivatives to remove high frequency artifacts.

At 304, the processing unit 120 determines velocity vectors based on the measured electrical signals. In some embodiments, to determine the velocity vectors the processing unit 120 determines relative activation times of the measured electrical signals at the location, fits a polynomial to the relative activation times, and calculates velocity vectors based on the polynomial.

At 306, the processing unit 120 determines or calculates activation times from the velocity vectors. In some embodiments, to determine the activation times from the velocity vectors, the processing unit 120 performs one or more of the following: interpolates the velocity vectors of each position of the multiple, different catheter positions to vertices of a mesh provided by the processing unit 120 as the geometry of the heart, creates a linear system of equations for the vertices of the mesh, and calculates an activation time at each of the vertices of the mesh by solving the linear system of equations.

Also, at least optionally, at 308, the processing unit 120 displays characteristics of the electrical activity of the heart. In some embodiments, the processing unit 120 displays one or more of: the degree of organization of the electrical signals at different locations in the heart, velocity vectors, cycle lengths, and at least some of the determined activation times. In some embodiments, the processing unit 120 displays characteristics of the electrical activity of the heart on a map of the heart, such as on display 170, to visualize one or more of the characteristics on the endocardium surface of the heart.

As described above in the description at 302, in some embodiments, the processing unit 120 determines whether the measured electrical signals are organized at each of the multiple, different catheter locations, and the processing unit 120 proceeds with further processing of the signals only if the measured electrical signals at the location of the multiple, different catheter locations are organized.

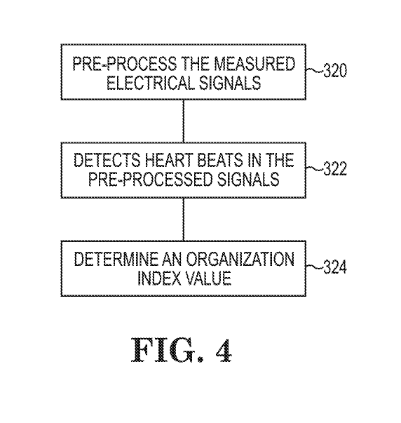

FIG. 4 is a flow chart diagram illustrating a process to determine whether the measured electrical signals at one of the positions of the catheter are organized, according to embodiments of the disclosure. This process can be performed by the cardiac mapping system 100, including the processing unit 120.

At 320, the processing unit 120, which may include the electrical module 140, pre-processes the measured electrical signals. In some embodiments, in pre-processing, the processing unit 120 provides one or more of: the processing unit 120 low pass filters the measured electrical signals to filter out high frequency noise, the processing unit 120 determines the derivatives of the low pass filtered signal, the processing unit 120 determines an absolute value of the derivatives of the low pass filtered signal to make the processed signals positive, and then, optionally, the processing unit 120 low pass filters the derivatives to remove high frequency artifacts.

At 322, the processing unit 120 detects heart beats in the pre-processed signals. In some embodiments, the processing unit 120 provides an adaptive threshold beat detection process for identifying beats and finding the threshold value that identifies beats and signals that are the most organized.

In some embodiments, the adaptive threshold process includes starting with a threshold value equal to some value, such as one half, of the highest electrical signal value. The threshold value is compared to the pre-processed signal, and points on the signal that meet or exceed the threshold value are identified as beat signals that indicate the beginning/ending of a beat. In this process, a 50 millisecond refractory period is provided around each identified beat signal for ignoring other possible beat signals and/or ignoring an upward change in the observed signal. Next, the intervals, also referred to as beat intervals, between adjacent beat signals are determined and, at 324, an organization index value is determined for the measured electrical signals.

In the adaptive threshold process, after each threshold value is processed to identify beat signals, the threshold value is adjusted and the process repeated. For, example, in an iterative process, the threshold value may be adjusted to one third, then one fourth, then one sixth, and then one eighth of the highest signal value, where intervals between adjacent beat signals are determined and an organization index is calculated for each of the different threshold values. The threshold value that provides the highest organization index value identifies the most organized set of beat signals and intervals, which are used for further processing and determining the velocity vectors and activation times of the electrical signals.

At 324, the processing unit 120 determines the organization index for each of the different threshold values. In embodiments, the processing unit determines the organization index value based on the variability of the beat intervals. For example, beat interval variability can be determined by subtracting a next beat interval value from the current beat interval value and dividing the subtraction result by the current beat interval value. The resulting value is the beat interval variability of the current beat interval. In some embodiments, the absolute value of the resulting value is the beat interval variability of the current beat interval.

Next, for a given length of the measured electrical signal, such as 2.5 seconds, the processing unit 120 determines the number of beat intervals that have a beat interval variability of less than a certain value or percentage, such as 15 percent. Also, in some embodiments, to be counted, each of the beat intervals should be between 100 milliseconds and 400 milliseconds in length.

Next, if at least a given number of beat intervals, such as eight beat intervals, in the measured electrical signal have a beat interval variability of less than the specified percentage, such as 15 percent, an organization index value is determined. The organization index value is calculated to be equal to the number of beat intervals with a beat interval variability of less than the percentage, such as 15 percent, divided by the total number of beat intervals detected in the sample of the measured electrical signal.

Next, the organization index value is compared to an organization index threshold or criteria to determine if the measured electrical signals are organized. In some embodiments, the organization index threshold is 0.6 or 60%. In other embodiments, a different organization index value may be chosen. In some embodiments, cycle length is determined. In some embodiments the cycle length is determined to be the median of the beat intervals that have a beat interval variability of less than the certain percentage, such as 15 percent.

As described above in the description at 302, in some embodiments, the measured electrical signals are assumed to be organized and the processing unit 120 pre-processes the measured electrical signals prior to further processing of the signals. Also, as described above in the process to determine whether the measured electrical signals at one of the positions of the catheter are organized at 320, the processing unit 120, which may include the electrical module 140, pre-processes the measured electrical signals.

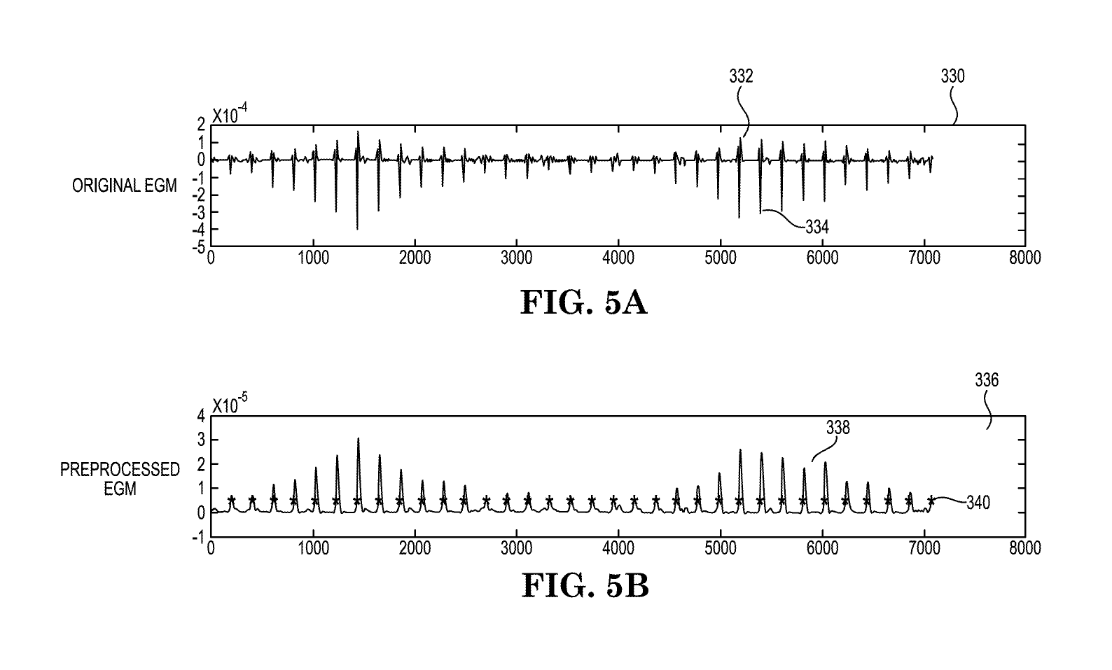

FIG. 5A is a diagram illustrating an original electrogram 330 of one of the measured electrical signals prior to pre-processing the measured electrical signal, according to embodiments of the disclosure. The original electrogram 330 includes values that are greater than zero at 332 and less than zero at 334. In addition, the original electrogram 330 includes a number of high frequency components, which could make it difficult to detect beat signals.

In embodiments, the original electrogram 330 is low pass filtered to remove or reduce high frequency noise. The processing unit 120 then determines the derivative of the low pass filtered signal and an absolute value of the derivative to make the processed signal positive. The absolute value of the derivative is then low pass filtered to further remove or reduce high frequency artifacts.

The resulting signal is illustrated in FIG. 5B, which is a diagram illustrating a pre-processed electrogram 336, according to embodiments of the disclosure. The pre-processed electrogram includes positive values at 338. In some embodiments, threshold values 340 can be compared to the pre-processed electrogram 336 to determine beat signals and beat intervals or cycle lengths. Also, in some embodiments, threshold values 340 can be compared to the pre-processed electrogram 336 to determine beat signals and beat intervals for determining the organization index value for the measured electrical signal.

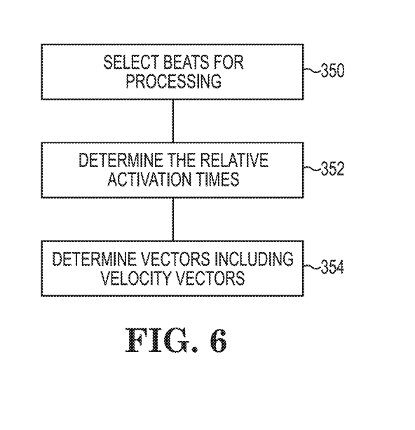

FIG. 6 is a flow chart diagram illustrating a process for determining relative activation times of the measured electrical signals and for determining vectors, such as propagation vectors and velocity vectors, at locations of the multiple, different catheter locations, according to embodiments of the disclosure.

At 350, the processing unit 120 selects beats for processing. Beats are selected based on one or more of the following criteria: the organization index value of the measured electrical signals that measured the beat; the number of electrodes that measured the beat and the positions of the electrodes in relation to the endocardium surface of the heart; and the positions of the electrodes that measured the beat in relation to one another on the splines of the catheter. In some embodiments, a beat must meet all of the above criteria before being selected for processing.

In some embodiments, to select a beat, the organization index value of the measured electrical signals of the beat must be greater than an organization index threshold value. In some embodiments the organization index threshold is 0.6 or 60%.

In some embodiments, to select a beat, the beat must be measured by at least a certain number of electrodes, such as 4 electrodes, and the electrodes must be within a certain distance, such as 2 or 3 millimeters, of the endocardium surface of the heart. This ensures the quality and integrity of the measured electrical signals.

In some embodiments, to select a beat, the electrodes that measured the beat must be on at least two splines of the catheter, such as catheter 210 of FIGS. 2A-2C. In some embodiments, to select a beat, the electrodes that measured the beat must be on at least two neighboring or adjacent splines of the catheter, such as catheter 210 of FIGS. 2A-2C.

At 352, the processing unit 120 determines the relative activation times at the electrodes that measured the beat. To determine the relative activation times, the processing unit 120 selects an electrogram interval around the selected beat for processing. In some embodiments, the processing unit 120 selects an electrogram interval of plus 75 milliseconds and minus 75 milliseconds around the beat.

Then, the processing unit 120 evaluates the measured electrical signals or electrograms of the beat to reduce or eliminate the number of noisy electrograms, and the processing unit 120 pre-processes the electrograms. In some embodiments, the processing unit 120 pre-processes the electrograms similar to the pre-processing of the measured electrical signals described above. The processing unit 120 performs one or more of: the processing unit 120 low pass filters the electrograms to filter out high frequency noise, the processing unit 120 determines the derivatives of the low pass filtered signal, the processing unit 120 determines an absolute value of the derivatives of the low pass filtered signal to make the processed signals positive, and, optionally, the processing unit 120 low pass filters the derivatives to remove high frequency artifacts. In some embodiments, the processing unit 120 performs all of the above steps in pre-processing the electrograms. In some embodiments, the processing unit 120 also removes the baseline of the electrograms.

Next, to determine the relative activation times, the processing unit 120 cross-correlates each of the pre-processed signals to each of the other neighboring pre-processed signals. The cross-correlation waveforms are evaluated, such as by the processing unit 120, to eliminate not useful or bad cross-correlation results, which may be cross-correlations that do not show a maximum or high correlation value. The cross-correlations are provided with respect to time, such that the location of the maximum correlation between two signals indicates the relative activation time between the two electrodes that measured the signals. In some embodiments, the processing unit 120 cross-correlates at least 4 signals from at least 4 electrodes that measured the selected beat.

At 354, the processing unit 120 determines vectors, such as propagation vectors and velocity vectors, at locations of the multiple, different catheter locations. First, the processing unit 120 projects the electrodes from the catheter, such as catheter 110 or catheter 210, onto a two-dimensional plane, where the processing unit 120 can also project the relative activations times of at least some of the electrodes onto the two-dimensional plane. The processing unit 120 then fits a polynomial, such as a first order polynomial, to the relative activation times. Based on the time gradients as depicted by the polynomial, the processing unit 120 calculates one or more of propagation vectors that are normalized and show the direction of propagation of the electrical activity and velocity vectors that indicate an amplitude or magnitude of the velocity vector and the direction of the activation of the electrical activity. As a result, in some embodiments, the mapping system has a velocity vector for each of the electrodes within a certain distance of the endocardium surface of the heart, which measured the beats.

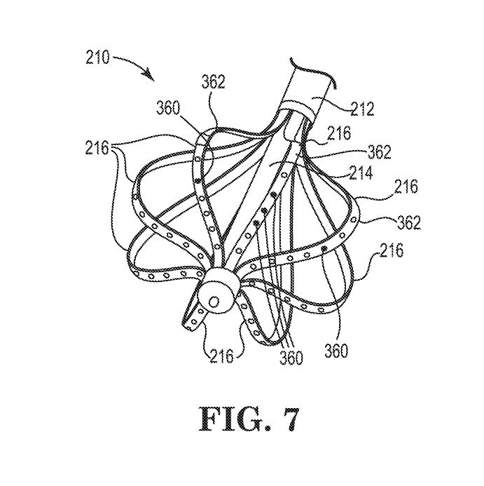

FIG. 7 is a diagram illustrating catheter 210 having five electrodes 360 on three different splines 362, which measure the electrical signals of a beat or beats of interest, according to embodiments of the disclosure. In this example, each of the five electrodes 360 is 1.5-2.5 millimeters from the endocardium surface when it measures the electrical signals of the heart. Also, the organization index value of the measured electrical signals has been found to be 70%.

In this example, the criteria for selection of a beat are as follows: the organization index value of the measured electrical signals that measured the beat must be greater than the organization index threshold of 60%; the beat must be measured by at least 4 electrodes that are within 3 millimeters of the endocardium surface of the heart; and the electrodes that measure the beat must be on at least two neighboring or adjacent splines of the catheter 210.

As described above, the organization index value of the measured electrical signals was found to be 70%, which satisfies the organization index threshold of 60%. Further, the electrical signals of the beat were measured by five different electrodes 360, which satisfies the number of electrodes criteria of at least 4 electrodes. Also, each of the five electrodes 360 was 1.5-2.5 millimeters from the endocardium surface of the heart, which satisfies the measuring distance criteria of being within 3 millimeters of the endocardium surface. In addition, the electrodes 360 are located on three different splines 304, where each of the splines is adjacent to or neighboring one of the other splines 304, which satisfies the at least two different neighboring spline criteria.

Thus, the five electrodes 360 on three different splines 362 that measure the electrical signals of the beat or beats of interest, in this example, satisfy the criteria for selection of a beat, such that the beat is selected by the processing unit 120.

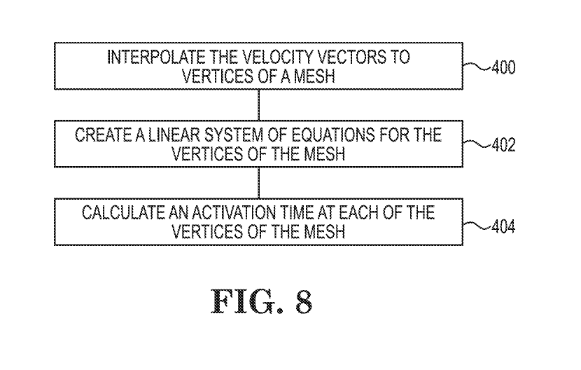

FIG. 8 is a flow chart diagram illustrating a process for determining activation times from the velocity vectors, according to embodiments of the disclosure.

At 400, to determine the activation times from the velocity vectors the processing unit 120 interpolates the velocity vectors to vertices of a mesh that is provided by the processing unit 120 as the geometry of the heart. As a result, in some embodiments, at least some of the vertices of the mesh have a velocity vector associated with it, and in some embodiments, each vertex of the mesh has a velocity vector associated with it.

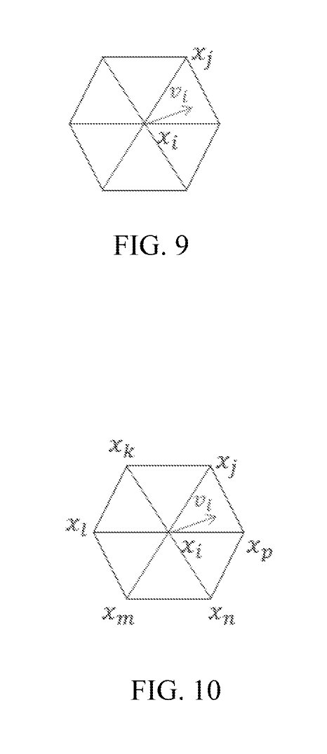

At 402, the processing unit 120 creates a linear system of equations for the vertices of the mesh. The processing unit 120 determines equations for neighboring vertices of the mesh, which relates activation times of neighboring vertices to each other. FIG. 9 is a diagram illustrating two neighboring points or vertices xi and xj on the mesh and a velocity vector vi at the point xi, according to embodiments of the disclosure. For any two neighboring points xi and xj on the mesh:

$${v}_{ij} = \frac{\vec{( {x}_{j} - {x}_{i} )} \cdot \vec{{v}_{i}}}{‖ {x}_{i} - {x}_{j} ‖}$$

$$where$$

$${v}_{ij} is the propagation speed in the direction of {x}_{i} to {x}_{j} and$$

$$\vec{{v}_{i}} is the estimated wavefront velocity at {x}_{i}$$

and the denominator represents the norm distance. Also,

$${t}_{j} - {t}_{i} = \frac{‖ {x}_{i} - {x}_{j} ‖}{{v}_{ij}}$$

$$where {t}_{i} and {t}_{j} are the activation times at {x}_{i} and {x}_{j}$$

To create the linear system of equations for the vertices of the mesh, the processing unit combines these equations for neighboring vertices of the mesh. Writing the above equations at each vertex of the mesh creates the linear system of equations as follows:
DT=V where T has the activation times at different vertices which are unknown and D and V are known

Also, D is an overdetermined matrix of all coefficients.

Next, at 404, the processing unit 120 calculates an activation time at each of the vertices of the mesh by solving the linear system of equations. An arbitrary activation time, such as zero, can be assigned to one of the vertices in order to solve the system of equations. Where, the processing unit 120 solves the linear system of equations using the overdetermined matrix of the linear system of equations as follows:

T=(D^{T}D)⁻¹DV

This results in determining the activation times at each vertex of the mesh.

In some embodiments, the processing unit 120 calculates an activation time at each of the vertices of the mesh by iteratively solving the linear system of equations. This iterative method is suitable for determining the activation times in reentrant cardiac rhythms.

FIG. 10 is a diagram illustrating neighboring points or vertices xi-xn and xp on a mesh and a velocity vector vi at the point xi, according to embodiments of the disclosure. The system of equations is calculated or determined as described above and to solve the system of equations iteratively, an arbitrary activation time, such as zero, can be assigned to one of the vertices, such as vertex xi. The processing unit 120 then solves the system of equations iteratively by finding activation times of each of the neighboring vertices of a vertex using the corresponding equations for the vertices. At each of the iterations, the processing unit 120 calculates the activation times of one neighboring layer of vertices of the vertex. For example, in the first iteration, the processing unit 120 finds the activation times of each of the neighboring vertices xj-xn and xp of vertex xi. In subsequent iterations, the processing unit 120 finds the activation times of vertices that neighbor each of the vertices xj-xn and xp, until the processing unit 120 has found activation times for all (or most) of the vertices of the mesh. At that point all of the activation times on the mesh are known.

Also, at least optionally, the processing unit 120 can display characteristics of the electrical activity of the heart. In some embodiments, the processing unit 120 displays one or more of: the degree of organization of the electrical signals at different locations in the heart, velocity vectors, cycle lengths, and at least some of the determined activation times. In some embodiments, the processing unit 120 displays characteristics of the electrical activity of the heart on a map of the heart, such as on display 170, to visualize one or more of the characteristics on the endocardium surface of the heart.

Various modifications and additions can be made to the exemplary embodiments discussed without departing from the scope of the disclosure. For example, while the embodiments described above refer to particular features, the scope of this disclosure also includes embodiments having different combinations of features and embodiments that do not include all of the described features. Accordingly, the scope of the disclosure is intended to embrace all such alternatives, modifications, and variations as fall within the scope of the claims, together with all equivalents thereof.

I claim:

1. A mapping system, comprising:
a catheter including multiple, spatially distributed electrodes configured to measure electrical signals of a heart;
a system configured to determine a position of the electrodes in the heart at multiple, different catheter positions in the heart; and
a processing unit configured to receive the measured electrical signals from each position of the multiple, different catheter positions, determine velocity vectors based on the measured electrical signals, and calculate, from the velocity vectors, an activation time at vertices of a mesh provided by the processing unit as a geometry of the heart.

2. The mapping system of claim 1, wherein to calculate, from the velocity vectors, an activation time at vertices of a mesh, the processing unit interpolates the velocity vectors of each position of the multiple, different catheter positions to the vertices of the mesh.

3. The mapping system of claim 1, wherein to calculate, from the velocity vectors, an activation time at vertices of a mesh, the processing unit creates a linear system of equations for the vertices of the mesh.

4. The mapping system of claim 3, wherein to create a linear system of equations for the vertices of the mesh, the processing unit determines equations for neighboring vertices of the mesh, which relate activation times of neighboring vertices to each other.

5. The mapping system of claim 4, wherein to create the linear system of equations for the vertices of the mesh, the processing unit combines the equations for neighboring vertices of the mesh to create the linear system of equations.

6. The mapping system of claim 3, wherein to calculate, from the velocity vectors, an activation time at vertices of a mesh, the processing unit solves the linear system of equations using an overdetermined matrix of the linear system of equations.

7. The mapping system of claim 3, wherein to calculate, from the velocity vectors, an activation time at vertices of a mesh, the processing unit solves the linear system of equations iteratively by finding activation times of each of the neighboring vertices of a vertex using corresponding equations for the vertices from the linear system of equations, wherein at each of the iterations, the processing unit calculates the activation times of one neighboring layer of vertices of the vertex.

8. The mapping system of claim 1, wherein to determine velocity vectors based on the measured electrical signals, the processing unit is configured to determine relative activation times of the measured electrical signals at the position, fit a polynomial to the relative activation times, and calculate the velocity vectors based on the polynomial.

9. The mapping system of claim 8, wherein:
to determine relative activation times of the measured electrical signals at the position, the processing unit is configured to:
select beats based on the position of the electrodes in the heart;
pre-process the beats selected via low pass filtering and determining absolute value;
cross-correlate pre-processed beats to neighboring pre-processed beats; and
determine the relative activation times based on locations of maximum correlation, and
to fit a polynomial to the relative activation times, wherein the processor is configured to project neighboring electrodes to a plane and fit a first order polynomial to the relative activation times.

10. The mapping system of claim 1, wherein to determine velocity vectors based on the measured electrical signals, the processing unit is configured to determine whether the measured electrical signals at a position of the catheter are organized, and if the measured electrical signals at the position are organized the processing unit is configured to determine relative activation times of the measured electrical signals at the position, fit a polynomial to the relative activation times, and calculate velocity vectors based on the polynomial.

11. The mapping system of claim 1, comprising displaying at least one of the velocity vectors and at least some of the activation times on a map of an endocardial surface of the heart.

12. A method of mapping electrical activity in a heart, comprising:
measuring electrical signals of the heart via a catheter including multiple, spatially distributed electrodes;
determining a position of the electrodes at multiple, different catheter positions in the heart;
measuring the electrical signals at each position of the multiple, different catheter positions;
determining, via a processing unit, velocity vectors based on the measured electrical signals; and
calculating, via a processing unit and from the velocity vectors, an activation time at vertices of a mesh provided by the processing unit as a geometry of the heart.

13. The method of claim 12, comprising interpolating the velocity vectors of each position of the multiple, different catheter positions to the vertices of the mesh and creating a linear system of equations for the vertices of the mesh.

14. The method of claim 12, wherein creating a linear system of equations for the vertices of the mesh includes determining equations for neighboring vertices of the mesh, which relate activation times of neighboring vertices to each other and combining the equations for neighboring vertices of the mesh to create the linear system of equations.

15. The method of claim 12, wherein calculating, via a processing unit and from the velocity vectors, an activation time at vertices of a mesh includes solving the linear system of equations using an overdetermined matrix of the linear system of equations.

16. The method of claim 12, wherein determining velocity vectors based on the measured electrical signals includes:
determining relative activation times of the measured electrical signals at the position;
fitting a polynomial to the relative activation times; and
calculating the velocity vectors based on the polynomial.

17. The method of claim 16, wherein:
determining relative activation times of the measured electrical signals at the position includes:
selecting beats based on the position of the electrodes in the heart;
pre-processing the beats selected via low pass filtering and determining absolute value;
cross-correlating pre-processed beats to neighboring pre-processed beats; and
determining the relative activation times based on locations of maximum correlation; and
fitting a polynomial to the relative activation times includes projecting neighboring electrodes to a plane and fitting a first order polynomial to the relative activation times.

18. A method of mapping electrical activity in a heart, comprising:
measuring electrical signals of the heart via a catheter including multiple, spatially distributed electrodes;
determining a position of the electrodes at multiple, different catheter positions in the heart;
measuring the electrical signals at each position of the multiple, different catheter positions;
determining, via a processing unit, velocity vectors based on the measured electrical signals;
interpolating the velocity vectors to vertices of a mesh provided by the processing unit as a geometry of the heart;
creating, via the processing unit, a linear system of equations for the vertices of the mesh by:
determining equations that relate activation times of neighboring vertices of the mesh to each other; and
combining the equations for neighboring vertices of the mesh to create the linear system of equations; and
calculating an activation time at each of the vertices of the mesh by solving the linear system of equations.

19. The method of claim 18, wherein determining velocity vectors based on the measured electrical signals includes:
determining relative activation times of the measured electrical signals at the position;
fitting a polynomial to the relative activation times; and
calculating the velocity vectors based on the polynomial.

20. The method of claim 18, comprising:
displaying the velocity vectors and at least some of the activation times on a map of the heart to visualize the velocity vectors and the at least some of the activation times on an endocardial surface of the heart.
